(19) United States
(12) Patent Application Publication
(10) Pub. No.: US 2025/0025285 A1
(43) Pub. Date: Jan. 23, 2025

(54) 3D PRINTED SCAFFOLDS FOR USE IN TISSUE REPAIR

(71) Applicant: AEVUMED, INC., Malvern, PA (US)

(72) Inventors: Saif Khalil, Malvern, PA (US); Miles Curtis, Berwyn, PA (US); Robert P. Douglass, Bryn Mawr, PA (US)

(21) Appl. No.: 18/789,081

(22) Filed: Jul. 30, 2024

Related U.S. Application Data
(63) Continuation-in-part of application No. 18/512,727, filed on Nov. 17, 2023.
(60) Provisional application No. 63/384,339, filed on Nov. 18, 2022.

(51) Int. Cl. A61F 2/08; A61L 27/36

(52) U.S. Cl. CPC A61F 2/08; A61L 27/3604; A61L 2430/10

(57) ABSTRACT

A biocompatible scaffold is described. The scaffold includes a synthetic polymer substrate having a geometry with a plurality of openings, and a natural polymer material integrated with the synthetic polymer substrate. The natural polymer material at least partially fills one or more of the openings in the synthetic polymer substrate.

### CROSS-REFERENCE TO RELATED APPLICATIONS

This application is a continuation-in-part of U.S. patent application Ser. No. 18/512,727, which claims priority to U.S. provisional application No. 63/384,339 filed Nov. 18, 2022, each of which is incorporated herein by reference in their entirety.

### BACKGROUND OF THE INVENTION

Soft tissue tears are common and many repair methods rely solely on the mechanical attributes of fasteners to repair the soft tissue. However, the fasteners alone are often inadequate for proper healing, as the soft tissue is in a weakened state and penetrating the soft tissue with the fasteners merely introduces additional weak points that are prone to further tearing.

3D printing is an emerging manufacturing technique that offers great precision to control the architecture of implantable scaffolds. Based on computer-aided design (CAD) models, 3D printers can fabricate a biocompatible construct in a layer-by-layer fashion. 3D printing and rapid prototyping processes have been used to create scaffolds with user defined micro-structures and micro-scaled architectures. However, these systems still lack higher sophistication both in the ability to control and define scaffold architecture.

Thus, there is a need in the art for improved scaffolds to utilize with anchoring systems to enhance repair and healing. The present invention meets this need.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an image of an exemplary 3D printing system depositing material in formation of a scaffold layer according to an aspect of the present invention.

FIGS. 2A and 2B are a schematic of exemplary scaffolds having two or more regions within the scaffold.

FIG. 3 is a schematic of an exemplary scaffold and suture anchoring device with a scaffold loaded into the device.

FIG. 4 is an image of a scaffold being positioned onto soft tissue via the device of FIG. 3.

FIG. 5 is an image of the positioned scaffold onto the soft tissue and between the soft tissue and bone.

FIG. 6 is an image of the scaffold being secured to the soft tissue and bone via bone anchors and sutures.

FIG. 7 is a flow diagram of an exemplary process of making a scaffold having a synthetic material structure and an integrated natural material.

FIG. 8 is an image of an exemplary 3D printed synthetic polymer substrate of a scaffold.

FIG. 9 is an image of an exemplary mold used to add a polymeric material to form a scaffold.

FIG. 10 is an image of an exemplary synthetic polymer substrate positioned in a mold.

FIG. 11 is an image of an exemplary synthetic polymer substrate before adding a natural polymer material (left), and an exemplary synthetic polymer substrate after adding a natural polymer material (right) to form a scaffold.

FIG. 12 is an image of collagen after it has been integrated to the synthetic polymer substrate by molding.

FIG. 13 is an image of collagen after it has been integrated to the synthetic polymer substrate by molding.

FIG. 14 is an image of collagen after it has been integrated to the synthetic polymer substrate by molding.

FIG. 15 is an image of collagen after it has been integrated to the synthetic polymer substrate by molding.

### SUMMARY OF THE INVENTION

Aspects of the invention relate to a biocompatible scaffold having a synthetic polymer substrate having a geometry with a plurality of openings, and a natural polymer material that is integrated with the synthetic polymer substrate and that at least partially fills one or more of the openings in the synthetic polymer substrate.

In some embodiments, the synthetic polymer substrate of the scaffold is the shape of a mesh, a meshwork, a grid, or a 3D geometry with a plurality openings that form thru-holes.

In some embodiments, the natural polymer material of the scaffold encapsulates the synthetic polymer substrate.

In some embodiments, the integrated natural polymer material is a molded layer, a 3D printed layer, an electrocompacted layer, or any combination thereof.

In some embodiments, the synthetic polymer of the scaffold is selected form the group consisting of: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), poly(L-lactic acid) (PLLA), polyglycolic acids (PGA), poly(lactide-co-glycolides) (PLGA), polycaprolactone (PCL), tri-calcium phosphate (TCP), polycaprilactone-tri-calcium phosphate (PCL-TCP), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO), polyorthoesters, and any combinations thereof.

In some embodiments, the natural polymer of the scaffold is selected from the group consisting of: collagen, fibrin, fibrinogen, thrombin, elastin, laminin, fibronectin, hyaluronic acid, chondroitin 4-sulfate, chondroitin 6-sulfate, dermatan sulfate, gelatin, heparin sulfate, heparin, keratan sulfate, proteoglycans, polysaccharides, chitin, chitosan, alginic acids, alginates, and any combinations thereof.

In some embodiments, the porosity of the scaffold is at least 80%.

Aspects of the invention relate to a method of making a biocompatible scaffold including the step of forming a synthetic polymer substrate having a plurality of openings, and the step of integrating a natural polymer material into one or more of the openings in the synthetic polymer substrate.

In some embodiments, the step of forming a synthetic polymer substrate includes molding or 3D printing.

In some embodiments, the method of making a biocompatible scaffold also includes the step of lyophilizing the synthetic polymer substrate and integrated natural polymer material.

In some embodiments, the step of integrating a natural polymer material into one or more openings in the synthetic polymer substrate includes molding, 3D printing, or electrocompacting.

In some embodiments, the step of integrating a natural polymer material into one or more openings in the synthetic polymer substrate includes encapsulating the synthetic polymer substrate within the natural polymer material.

In some embodiments, the step of forming a synthetic polymer substrate includes forming geometry that is a mesh, a meshwork, a grid, or a 3D geometry with thru-holes.

In some embodiments, the method of making a biocompatible scaffold also includes the steps of positioning the synthetic polymer substrate into a mold, mixing a buffer solution with the natural polymer material to form a gel, adding the gel into the mold containing the synthetic polymer substrate, curing the gel, freezing the synthetic polymer substrate and gel, and lyophilizing the frozen synthetic polymer substrate and gel to form the scaffold.

### DETAILED DESCRIPTION

### Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, for example numerical values and/or ranges, such as an amount, a temporal duration, and the like, is meant to encompass variations of +20%, +10%, +5%, +1%, or +0.1% from the specified value, as such variations are appropriate to perform the disclosed methods. For example, “about 40 [units]” may mean within +25% of 40 (e.g., from 30 to 50), within +20%, +15%, +10%, +9%, +8%, +7%, +6%, +5%, +4%, +3%, +2%, +1%, less than +1%, or any other value or range of values therein or therebelow. Furthermore, the phrases “less than about [a value]” or “greater than about [a value]” should be understood in view of the definition of the term “about” provided herein.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

The present invention relates in part to biocompatible and implantable scaffolds. In some embodiments, the scaffolds are tailored for tendon repair, including tendon-to-tendon and/or tendon-to-bone applications. In some embodiments, the scaffolds form homogenous or non-homogenous, single layer or multi-layered constructs. The present invention also relates in part to methods for depositing natural and/or synthetic polymers to create any of the biocompatible and implantable scaffolds described herein. In some embodiments, the scaffolds are a combination of any number of synthetic material layers and natural material layers. In some embodiments, the scaffolds have a synthetic material structure that is formed of one or more synthetic material layers. Individual synthetic material layers or a portion of a synthetic material layer can be incorporated with or can be encapsulated by a natural material layer. In some embodiments, a whole or portion of a synthetic material structure formed of synthetic material layers is encapsulated by or integrated with a natural material layer. In some embodiments, the scaffolds have a synthetic material structure upon which a natural material layer is attached. In some embodiments, the scaffolds have any number of layers including structural layers, integrated layers, encapsulating layers, or coatings. In some embodiments, the scaffold is formed by 3D printing a synthetic material layer or structure and introducing a natural polymer to the synthetic polymer structure by molding. The natural polymer may encapsulate and/or be integrated with the synthetic polymer structure. The present invention also relates in part to methods for depositing natural and/or synthetic polymers to create any of the biocompatible and implantable scaffolds described herein.

Scaffolds composed of a combination of synthetic and natural polymer layers may be more robust to bending, twisting, tension, or compression than scaffolds composed of only natural polymers. Scaffolds composed of a combination of synthetic and natural polymer layers may be easier to handle, more amenable to manipulation or suturing, or less likely to break or degrade than scaffolds composed of only natural polymers.

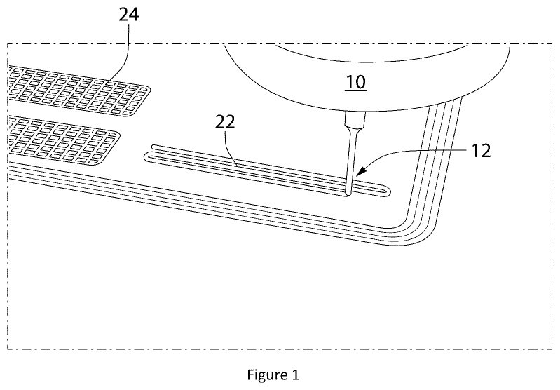

As contemplated herein, the scaffolds may be constructed from one or more layers of deposited material. The scaffolds may be constructed by single manufacturing techniques, or combinations of manufacturing techniques, including techniques such as 3D printing, molding, etching, sintering, or any other additive manufacturing techniques. FIG. 1 shows a 3D printer 10 having a deposition needle 12 with a distal tip opening sized to deposit the desired amount of material in either droplets or as a continuously flowing strand 22 to form one or more scaffold layers 24. In some embodiments, one or more scaffold layers are of a synthetic material. In some embodiments, one or more scaffold layers form a synthetic material structure or synthetic material substrate. In some embodiments, an additional support material is deposited onto or within the spacing of a scaffold layer or synthetic material structure. In some embodiments, such support materials may effectively fill the spacing between strands, or otherwise fully fill or at least partially fill the porosity of any previously deposited material in the scaffold or scaffold layer. In some embodiments, the support material may form part of the final scaffold structure or it may be removed from the final scaffold mechanically or chemically, for example via heating, cooling, drying, dissolving and the like. In some embodiments, the support material may form part of a 3D printed synthetic material scaffold layer or structure. In some embodiments, the support material may be a natural material fully or partially coating or encapsulating the synthetic material.

In some embodiments, the final scaffold structure may be generated by further incorporating a natural material layer with a 3D printed synthetic material scaffold layer or structure. In some embodiments, a natural polymer layer encapsulates at least a portion of a 3D printed synthetic material structure or a scaffold layer. In some embodiments, natural polymer layers are added by molding, 3D printing, or electrocompacting.

In some embodiments, certain individual strands of the scaffold or a scaffold layer may have an average diameter of less than 2 mm, less than 1.8 mm, less than 1.6 mm, less than 1.4 mm, less than 1.2 mm, less than 1 mm, less than 0.8 mm, less than 0.6 mm, less than 0.4 mm, less than 0.2 mm or less than 0.1 mm. In some embodiments, the average diameter of certain individual strands of the scaffold or a scaffold layer may have an average diameter of between 0.1 mm and 2 mm. In some embodiments, the strands of the scaffold or the strands of a scaffold layer have about the same average diameter. In some embodiments, the strands of the scaffold or the strands of a scaffold layer have a variable average diameter. For example, in some embodiments, 90% of the strands have an average diameter of less than 2 mm and 50% of the strands have an average diameter of less than 1 mm.

In some embodiments, the spacing between certain adjacent individual strands of the scaffold or a scaffold layer may have an average distance of less than 2 mm, less than 1.8 mm, less than 1.6 mm, less than 1.4 mm, less than 1.2 mm, less than 1 mm, less than 0.8 mm, less than 0.6 mm, less than 0.4 mm, less than 0.2 mm or less than 0.1 mm. In some embodiments, the average distance between certain adjacent individual strands of the scaffold or a scaffold layer may have an average distance of between 0.1 mm and 2 mm. In some embodiments, the adjacent strands of the scaffold or a scaffold layer have about the same average distance apart. In some embodiments, the adjacent strands of the scaffold or a scaffold layer have a variable average distance apart. For example, in some embodiments, 90% of adjacent strands have an average distance apart of less than 2 mm and 50% of adjacent strands have an average distance apart of less than 1 mm.

As mentioned previously, the scaffolds of the present invention may include one or more layers of deposited material. In some embodiments, the scaffold includes multiple regions, where a first region has more layers than a second region. In some embodiments, the strand orientation between layers may be between 0° and 90°, between 0° and 80°, between 0° and 70°, between 0° and 60°, between 0° and 50°, between 0° and 40°, between 0° and 30°, between 0° and 20°, between 0° and 10°, or between 0° and 5°. In some embodiments, the strand orientation between layers may be about 5°, about 10°, about 15°, about 20°, about 25°, about 30°, about 35°, about 40°, about 45°, about 50°, about 55°, about 60°, about 65°, about 70°, about 75°, about 80° or about 85°. In some embodiments, the strand orientation between layers may be substantially parallel. In some embodiments, the strand orientation between layers may be substantially perpendicular. In some embodiments, the strands may be oriented in a grid-like structure layer by layer. In some embodiments, each layer may have the same or a different strand diameter and spacing to an adjacent layer. In some embodiments, one or more layers may or may not include a support material. The support material for each layer may entirely fill or at least partially fill voids within the strands of one or more layers of the scaffold. In some embodiments, natural polymer layers are incorporated within individual synthetic polymer layers. In some embodiments, natural polymer layers fully fill or partially fill the voids of one or more synthetic polymer layers.

In various embodiments, scaffolds or scaffold layers of the present invention can have any desired shape, including but not limited to square, rectangular, polygonal, circular, ovoid, and irregularly shaped sheets. The scaffolds of the present invention may be rigid, flexible, clastic, or combinations thereof. For example, the scaffold may include multiple regions, where a first region is rigid or semi-rigid, and a second region is flexible, semi-flexible and/or elastic. In some embodiments, the scaffolds may include one or more hinging regions, such that the scaffold is configured to bend or fold.

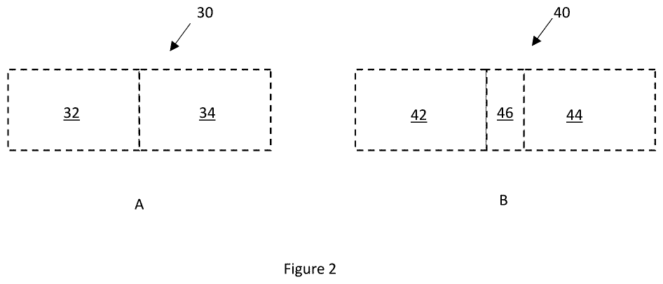

In some embodiments, the scaffold or any individual region thereof has an overall porosity of between 50% and 99.9%. In some embodiments, the scaffold has an overall porosity of about 50%, greater than 60%, greater than 70%, greater than 80%, greater than 90%, greater than 92%, greater than 94%, greater than 96%, greater than 98%, greater than 99%, greater than 99.2%, greater than 99.4%, greater than 99.6%, greater than 99.8% or about 99.9%. In some embodiments, the synthetic polymer substrate or individual region thereof, the natural polymer material or individual region thereof, or any particular layer of the scaffold or individual region thereof has an overall porosity of between 50% and 99.9%. In some embodiments, the particular scaffold layer has an overall porosity of about 50%, greater than 60%, greater than 70%, greater than 80%, greater than 90%, greater than 92%, greater than 94%, greater than 96%, greater than 98%, greater than 99%, greater than 99.2%, greater than 99.4%, greater than 99.6%, greater than 99.8% or about 99.9%. In some embodiments, the porosity of adjacent layers is the same or may be different. In some embodiments, the effective porosity of the scaffold may be the porosity of an incorporated natural material polymer. In some embodiments, the porosity of the scaffold is about 50%, about 60%, about 70%, about 80%, about 85%, about 90%, about 95%, about 98%, about 99%, or about 99.5%. In some embodiments, the scaffold porosity may be induced or enhanced via lyophilization. In some embodiments, the porosity of a synthetic material layer is different from the porosity of a natural material layer. In some embodiments, the scaffold, the synthetic polymer substrate, the natural polymer material, or any layer, may have different porosities in different regions. For example, as shown in FIG. 2A, scaffold 30 may have a first porosity in region 32 and a second, different porosity in region 34. Likewise, scaffold 40 of FIG. 2B may have a first porosity in region 42, a second porosity in region 44, and a third porosity in region 46.

In some embodiments, the scaffold or a scaffold layer may have a length of between about 1 mm and 50 mm, between about 2 mm and 50 mm, between about 5 mm and 50 mm, between about 1 mm and 40 mm, between about 2 mm and 40 mm, between about 5 mm and 40 mm, between about 10 mm and 30 mm or between about 10 mm and 20 mm. In some embodiments, the scaffold or a scaffold layer may have a length of less than 50 mm, less than 45 mm, less than 40 mm, less than 35 mm, less than 30 mm, less than 25 mm, less than 20 mm, less than 15 mm, less than 10 mm, less than 5 mm, less than 4 mm, less than 3 mm, less than 2 mm, or less than 1 mm.

In some embodiments, the scaffold or a scaffold layer may have a width of between about 1 mm and 50 mm, between about 2 mm and 50 mm, between about 5 mm and 50 mm, between about 1 mm and 40 mm, between about 2 mm and 40 mm, between about 5 mm and 40 mm, between about 10 mm and 30 mm or between about 10 mm and 20 mm. In some embodiments, the scaffold or a scaffold layer may have a width of less than 50 mm, less than 45 mm, less than 40 mm, less than 35 mm, less than 30 mm, less than 25 mm, less than 20 mm, less than 15 mm, less than 10 mm, less than 5 mm, less than 4 mm, less than 3 mm, less than 2 mm, or less than 1 mm.

In some embodiments, the scaffold or a scaffold layer may have a thickness of between about 0.01 mm and 5 mm, between about 0.02 mm and 5 mm, between about 0.05 mm and 5 mm, between about 0.01 mm and 4 mm, between about 0.02 mm and 4 mm, between about 0.05 mm and 4 mm, between about 0.1 mm and 3 mm or between about 0.1 mm and 2 mm. The thickness of any portion of the scaffold or a scaffold layer may be based on the material composition of the strands and/or support material. The thickness of any portion of the scaffold may be also based on the number of layers forming the scaffold. Each layer of the scaffold may have a thickness and is formed by 3D printing of one or more strands and/or support material or a layer of the scaffold may be formed by pouring a material into or on top of another layer such that gaps in a previous layer are filled or partially filled by the new material layer. In some embodiments, the scaffold or a scaffold layer may have a thickness of less than 5 mm, less than 4.5 mm, less than 4.0 mm, less than 3.5 mm, less than 3.0 mm, less than 2.5 mm, less than 2.0 mm, less than 1.5 mm, less than 1.0 mm, less than 0.5 mm, less than 0.4 mm, less than 0.3 mm, less than 0.2 mm, less than 0.1 mm, less than 0.09 mm, less than 0.08 mm, less than 0.07 mm, less than 0.06 mm, less than 0.05 mm, less than 0.04 mm, less than 0.03 mm, less than 0.02 mm, or less than 0.01 mm. In some embodiments, the scaffold or a scaffold layer has a uniform thickness. In some embodiments, the scaffold or a scaffold layer has a variable thickness.

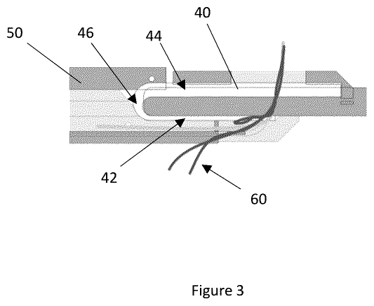

In some embodiments, the scaffold or a scaffold layer has a first region of a first thickness, and a second region of a second thickness. It should be appreciated that the scaffold or a scaffold layer may have any number of regions, each having a respective thickness and functional mechanical property (such as rigidity, flexibility, elasticity, material or mechanical strength, etc.). For example, as shown in FIG. 2A, the scaffold 30 may have a first region 32 having a first thickness, and a second region 34 having a second thickness, where the thickness of region 34 is greater than or equal to the thickness of region 32. Such exemplary scaffolds may be suitable for and configured to provide more or less mechanical strength based in part on the thickness of each region when implanted into a subject. Further, by having regions of greater or lesser thickness can promote better fit on top of or between certain tissues when implanted in a subject. In some embodiments, the thickness of the second region is at least 10% the thickness of the first region, at least 15% the thickness of the first region, at least 20% the thickness of the first region, at least 25% the thickness of the first region, at least 30% the thickness of the first region, at least 35% the thickness of the first region, at least 40% the thickness of the first region, at least 45% the thickness of the first region, at least 50% the thickness of the first region, at least 60% the thickness of the first region, at least 70% the thickness of the first region, at least 80% the thickness of the first region, at least 90% the thickness of the first region, at least 100% the thickness of the first region, or at least 200% the thickness of the first region. As shown in FIG. 2B, the scaffold 40 may have a first region 42 having a first thickness, a second region 44 having a second thickness, and a third region 46 having a third thickness, where the thickness of region 44 is greater than or equal to the thickness of region 42, and where the thickness of region 42 is greater than or equal to region 46. Exemplary scaffolds of FIG. 2B are shown positioned within a scaffold and suture anchoring device in FIG. 3, and may be configured to fold at region 46, while regions 42 and 44 are configured to accept and support one or more sutures passing through the scaffold and neighboring or adjacent biological tissues and/or bone anchors.

In some embodiments, the scaffold has first and second regions with different strand orientations. These regions can have the same thickness or different thicknesses. Variation in strand orientation can apply to scaffolds with any number of regions, each having a respective strand orientation that corresponds to at least one of a functional mechanical property (such as rigidity, flexibility, elasticity, material or mechanical strength, etc.), an anatomy of the patient, or the surgeons preferred implantation pattern, e.g. creating more strength where sutures pierce the scaffold, or creating a recessed area on the scaffold for sutures to rest in. For example, the scaffold may have a first region having a first strand orientation, and a second region having a second strand orientation, and the thickness of region could be the same or different than the thickness of region. Such exemplary scaffolds may be suitable for and configured to provide more or less mechanical strength based in part on the strand orientation of each region when implanted into a subject. The different strand orientations may also provide more rigidity or flexibility in certain directions, which depending on the anatomy of the patient and the implantation technique can promote better performing custom implant.

Scaffolds of the present invention can comprise synthetic materials, biological materials, and combinations thereof to enhance biocompatibility and healing. In some embodiments, strands, support material, integrating material, encapsulating material, and/or coating material of the scaffold may be composed of a single material. In some embodiments, strands, support material, integrating material, encapsulating material, and/or coating material of the scaffold may be composed of multiple materials. In some embodiments, the strands, support material, integrating material, encapsulating material, and/or coating material is a synthetic material. In some embodiments, the strands, support material, encapsulating material, and/or coating material is a biological material. In some embodiments, strands, support material, encapsulating material, and/or coating material is a composite. In some embodiments, the strands, support material, encapsulating material, and/or coating material is a combination of synthetic and biological materials. As contemplated herein, each layer of the scaffold may be formed of strands composed of one or more materials.

Contemplated synthetic strand, support materials, integrating materials, encapsulating materials, and/or coating materials include but are not limited to: poly(urethanes), poly(siloxanes) or silicones, poly(ethylene), polyN (vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), poly(L-lactic acid) (PLLA), polyglycolic acids (PGA), poly(lactide-co-glycolides) (PLGA), polycaprolactone (PCL), tri-calcium phosphate (TCP), polycaprilactone-tri-calcium phosphate (PCL-TCP), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO), polyorthoesters, any combinations thereof or any other similar synthetic polymers that may be developed that are biologically compatible. Contemplated biologic or natural strand, support materials, integrating materials, encapsulating materials, and/or coating materials such as proteins, peptides, polysaccharides, enzymes and cells may be included. Proteins, peptides, polysaccharides, enzymes, or cells may be of any origin including human, bovine, or porcine. Other examples of biologic or natural materials include but are not limited to: collagen Type I, collagen Type II, collagen Type III, collagen Type IV, collagen Type V, combination of collagen types (e.g. Type I with Type II, Type I with Type III, Type II with Type III, etc.), hyaluronic acid (HA), fibrin, fibrinogen, thrombin, elastin, laminin, fibronectin, hyaluronic acid, chondroitin 4-sulfate, chondroitin 6-sulfate, dermatan sulfate, gelatin, heparin sulfate, heparin, and keratan sulfate, proteoglycans, polysaccharides (e.g. cellulose and its derivatives), chitin, chitosan, alginic acids, and alginates such as calcium alginate and sodium alginate, and any combinations of biological and/or synthetic materials. In some embodiments, scaffolds of the present invention comprise tissue grafts. In some embodiments, scaffolds of the present invention comprise isotropic materials. In other embodiments, scaffolds of the present invention comprise anisotropic fibers, such that they can be positioned in a direction that aligns anisotropic fibers in a direction of natural or expected anatomic forces to resist tearing and further damage.

In some examples, a scaffold is made of a synthetic material structure or substrate and an integrated natural material. A synthetic material structure or layers of a synthetic material structure of a scaffold may be any shape and/or include strands of any properties. A synthetic material structure or layers of a synthetic material structure of a scaffold may also include any support materials. A synthetic material structure may be a 2-dimensional pattern with a single thickness or varying thickness. In some examples, the shape of the synthetic material structure is designed to promote the integration of a natural material layer for example by incorporating thru-holes. For example, the synthetic material structure may be a mesh, a meshwork, or a grid. In some examples, the synthetic material structure is a 3D geometry with a single height or varying height and may include thru-holes in one or more planes. In some examples, the thickness or height of the synthetic material structure or substrate in one or more regions may range from 10 μm to 2 mm. In some examples, the thickness of the synthetic material substrate in one or more regions may range from 50 μm to 1.5 mm. In some examples, the thickness of the synthetic material substrate in one or more regions may range from 100 μm to 1 mm. In some examples, the thickness of the synthetic material substrate in one or more regions may range from 150 μm to 800 μm. In some examples, the thickness of the synthetic material substrate in one or more regions may range from 200 μm to 600 μm. In some examples, the thickness of the synthetic material substrate in one or more regions may range from 200 μm to 500 μm. In some examples, the thickness of the synthetic material substrate in one or more regions may range from 200 μm to 400 μm. In some examples, the thickness of the synthetic material substrate in one or more regions may range from 200 μm to 300 μm. In some examples, the thickness of the synthetic material substrate in one or more regions may range from 500 μm to 1500 μm. In some examples, the thickness of the synthetic material substrate in one or more regions may range from 600 μm to 1000 μm.

In some examples, the thickness or height of the natural polymer material in one or more regions may range from 10 μm to 2 mm. In some examples, the thickness of the natural polymer material in one or more regions may range from 50 μm to 1.5 mm. In some examples, the thickness of the natural polymer material in one or more regions may range from 100 μm to 1 mm. In some examples, the thickness of the natural polymer material in one or more regions may range from 150 μm to 800 μm. In some examples, the thickness of the natural polymer material in one or more regions may range from 200 μm to 600 μm. In some examples, the thickness of the natural polymer material in one or more regions may range from 200 μm to 500 μm. In some examples, the thickness of the natural polymer material in one or more regions may range from 200 μm to 400 μm. In some examples, the thickness of the natural polymer material in one or more regions may range from 200 μm to 300 μm. In some examples, the thickness of the natural polymer material in one or more regions may range from 500 μm to 1500 μm. In some examples, the thickness of the natural polymer material in one or more regions may range from 600 μm to 1000 μm. In some examples, the thickness of the natural polymer material in one or more regions may range from 100 μm to 200 μm. In some examples, the thickness of the natural polymer material in one or more regions may range from 400 μm to 600 μm.

In some examples, the thickness or height of the overall scaffold in one or more regions may range from 10 μm to 2 mm. In some examples, the thickness of the overall scaffold in one or more regions may range from 50 μm to 1.5 mm. In some examples, the thickness of the overall scaffold in one or more regions may range from 100 μm to 1 mm. In some examples, the thickness of the overall scaffold in one or more regions may range from 150 μm to 800 μm. In some examples, the thickness of the overall scaffold in one or more regions may range from 200 μm to 600 μm. In some examples, the thickness of the overall scaffold in one or more regions may range from 200 μm to 500 μm. In some examples, the thickness of the overall scaffold in one or more regions may range from 200 μm to 400 μm. In some examples, the thickness of the overall scaffold in one or more regions may range from 200 μm to 300 μm. In some examples, the thickness of the overall scaffold in one or more regions may range from 500 μm to 1500 μm. In some examples, the thickness of the overall scaffold in one or more regions may range from 600 μm to 1000 μm.

As contemplated herein, the synthetic polymer substrate may have any desired geometry. In some examples, the synthetic material structure is a grid or 3D geometry with thru-holes having a rectangular base with some height. In some examples, the rectangular base has lengths of approximately 2 cm and 3 cm. In some embodiments, the synthetic material structure is a grid having openings with a characteristic length ranging from 10 μm to 1 mm. The openings may be any shape and size. In some embodiments, the synthetic material structure is grid having rectangular openings. In some examples, the rectangular openings of a synthetic material structure grid have side lengths ranging from 10 μm to 1 mm. In some embodiments, the rectangular openings are squares. In some embodiments, all grid openings have the same geometry. In alternative embodiments, the grid openings have a distribution of geometries. In some embodiments, the grid has smaller openings in some portions and larger openings in other portions. Synthetic material substrates or sections of synthetic material substrates with larger pores or grid openings may be weaker, while synthetic material substrates or sections with smaller pores or grid openings be stronger. Synthetic material substrates or sections of synthetic material substrates with a larger thickness may be stronger, while synthetic material substrates or sections of synthetic material substrates with a smaller thickness may be weaker. For example, the synthetic material substrate may have a rectangular base with a first and second section at each end of the rectangular base. One section may have a thickness that is smaller for example about 200 μm to 400 μm, and another section may have a thickness that is larger for example about 600 μm to 1 mm. In this embodiment, one end of the synthetic material substate may be stronger than the other end. In some examples, the synthetic material structure is a grid with rectangular base having side lengths of approximately 2 cm and 3 cm and rectangular openings that range that have side lengths ranging from approximately 400 μm to 600 μm. In some examples, the openings or porosity of the synthetic material structure are significantly larger than the openings or porosity of an integrated natural material polymer. For example, the porosity or geometry of openings of the synthetic material structure may be optimized for desired properties of scaffold strength, while the porosity of the integrated natural material may be optimized to promote the incorporation of cells within the scaffold. For example, the openings or pores of the synthetic material structure may range from having a characteristic length from 400 μm to 600 μm, and the openings or pores of the natural material polymer may range from having a characteristic length from 50 μm to 250 μm. In some examples, the synthetic material structure may be of a synthetic material or synthetic material polymer that promotes the integration of natural materials. For example, the synthetic material may have roughness properties that promote adhesion of natural materials.

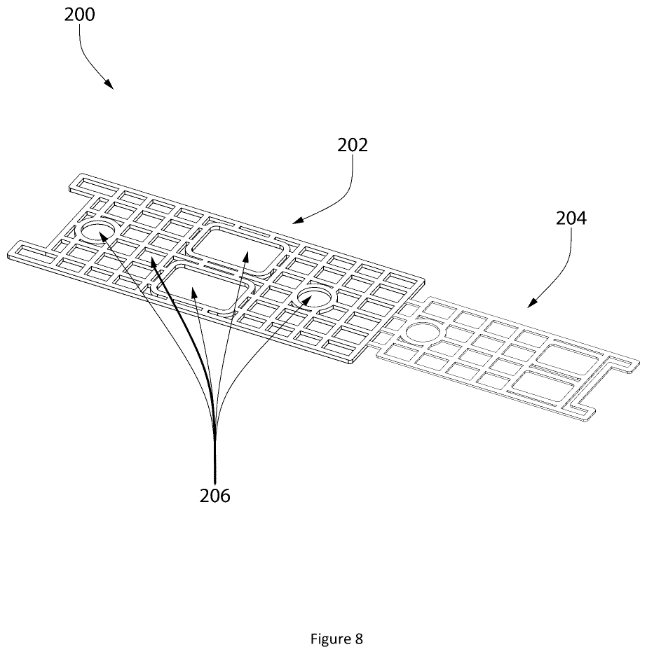

Now referring to FIG. 8, in some embodiments, a synthetic polymer substrate or structure has any number of sections. For example, as in FIG. 8, the synthetic polymer structure 200 has a first section 202 and a second section 204. In some examples, each section has a different thickness as in FIG. 8. In some examples, as in FIG. 8, the synthetic polymer substrate may be a grid with various shapes and sizes of openings 206. For example, as in FIG. 8, the openings can be of variable sizes. In some embodiments, the corners of the rectangular openings may be rounded. Rounded corners may promote natural polymer material integration with or natural polymer material adhesion to the synthetic material polymer substrate. In some embodiments, the area of larger grid openings may be 1.1 to 10 times larger than smaller grid openings. In some embodiments, the thickness of a section, for example section 202 or 204 is designed such that it is strong enough to be sutured without tearing. In some embodiments, the thickness of a section, for example section 202 or 204 is designed such that it is pliable enough to be easily manipulated when applied to a subject. In some embodiments, openings 206 of the synthetic polymer scaffold are designed such that they can fit into or over the support structure of a mold to increase the case of positioning within a mold and to increase the reproducibility of substrate positioning in the mold. For example, openings 206 may be designed to fit flush against a mold support.

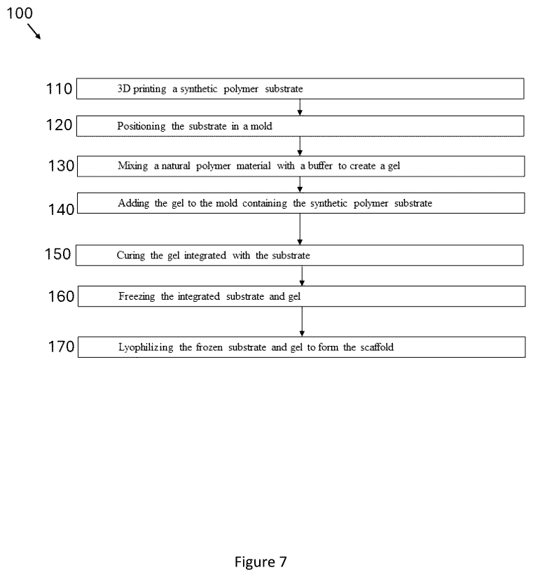

Aspects of the invention relate to various methods of making a scaffold. Now referring to FIG. 7, shown is an exemplary method 100 for making a scaffold having a synthetic material polymer structure and an integrated or encapsulating natural material. In some embodiments, method 100 comprises the steps of 110 3D printing a synthetic polymer substrate, 120 positioning the substrate in a mold, 130 mixing a natural polymer material with buffer to create a gel, 140 adding the gel to the mold containing the synthetic polymer substrate, 150 curing the gel integrated with the substrate, 160 freezing the integrated substrate and gel, and 170 lyophilizing the scaffold.

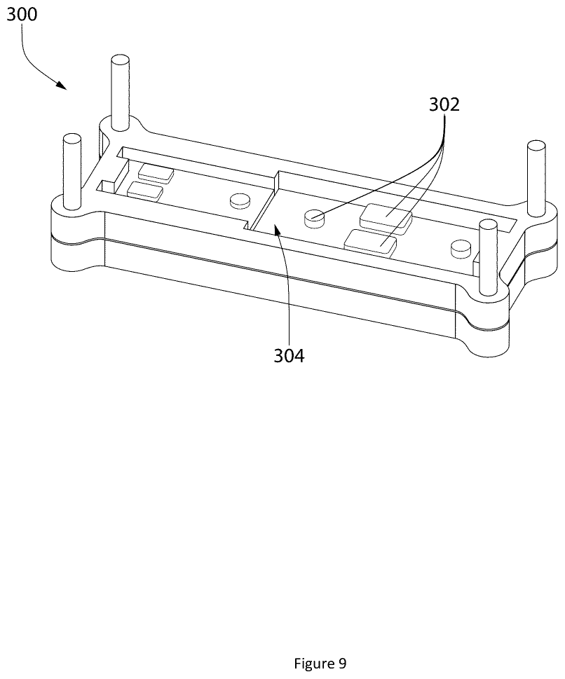

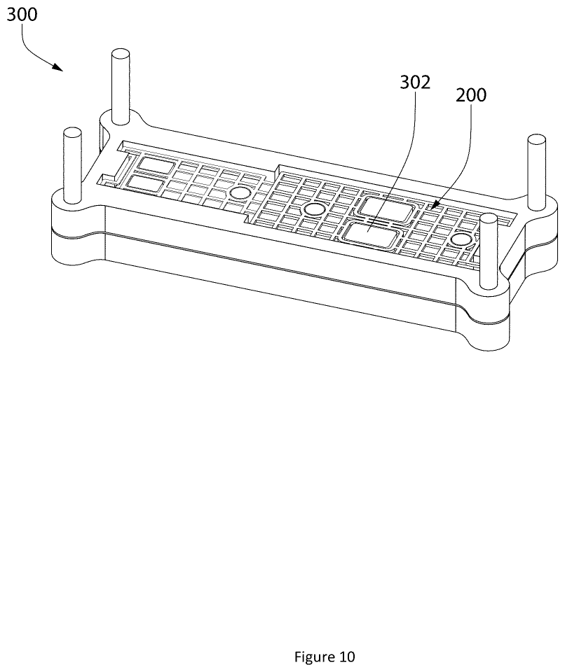

Various techniques may be used to place or position the synthetic material structure in the mold. In some embodiments, the mold may include support structures to position the synthetic material structure within the mold. Now referring to FIG. 9, the support structures 302 of a mold 300 may be designed such that they fit into openings 206 of the synthetic polymer substrate 200. The mold cavity 304 may be designed such that the mold cavity 304 thickness matches the desired thickness of the overall scaffold after the natural polymer material is added. Now referring to FIG. 10, the synthetic material structure 200 may be positioned so that it lays flat in the mold cavity 304. In some embodiments, the support structures 302 of the mold 300 are flush against the edges of openings 206 in the synthetic polymer substrate 200. In some embodiments, there is a spacing between the support structures 302 of the mold 300 and the edges of openings 206 in the synthetic polymer substrate 200 so that the position of the synthetic polymer structure 200 within the mold cavity 304 can be manipulated before a gel is poured. The synthetic material structure 200 may be placed into the mold 300 at any orientation. The mold 300 may be designed such that the synthetic polymer structure 200 is flush against the mold cavity 304 walls and floor so that when a natural material solution is poured into the mold cavity 304 it is only integrated within the synthetic material structure 200. In some embodiments, the mold 300 is designed such that there is spacing between the edges of the mold cavity 304 and the synthetic material structure 200 such that when a natural material solution is poured into the mold cavity 304 the synthetic material structure is fully encapsulated. For example, the mold 300 may be designed such that the natural material polymer encapsulates the substrate 200 and adds some thickness to the overall scaffold. In some examples, the thickness of the encapsulating natural material polymer is variable in different sections 202 and 204 or any other portion of the synthetic material polymer substrate 200. For example, the mold 300 may be designed with a spacing between the synthetic material structure 200 and the mold cavity 304 walls such that there are natural material protrusions in the scaffold after molding a natural material. In some embodiments, a barrier may be placed in the mold cavity 304 such that two or more natural material solutions can be poured into different regions.

Natural materials including natural polymers may be prepared in any manner before being incorporated with, integrated with, or encapsulating a synthetic material structure. The prepared natural materials may be poured into a mold over a synthetic material structure so that it can be incorporated into, integrated into, or encapsulate the structure. For example, natural materials may be prepared by mixing with a buffer solution to create a gel. The duration of mixing, the temperature and pH at which mixing occurs, and the concentration of the natural material in the buffer solution may be optimized for desired natural material and polymer properties or desired material properties of the solution after gelling, including metrics of polymer length distribution, polymer crosslinking, polymer density, polymer crowding, material stiffness, material viscoelasticity, and/or porosity.

Various techniques may be used to pour a natural material gel into the mold. In some embodiments, the gel is de-gassed before being introduced to the mold. In some embodiments, the gel is de-gassed after being introduced to the mold. In some embodiments, the gel is poured into the mold with a pipettor. In some embodiments, the volume of gel poured into the mold is optimized to achieve the desired final scaffold volume. In some embodiments, some gel is poured into the mold and optionally allowed to solidify or cure, then a 3D printed synthetic material structure is placed on the gel before pouring the rest of the gel. In this embodiment, a 3D printed synthetic material structure may be sandwiched between natural polymer material layers. In some embodiments, the mold is coated with a material that prevents adherence to the gel, for example a hydrophobic material.

In some embodiments, the synthetic material structure and the gel are incubated in the mold for any duration and at any temperature, pressure, or humidity. The incubation may promote the partial or full solidification and/or curing of the gel. The incubation may promote the integration or incorporation of the gel into the synthetic material structure. The temperature may be optimized for desired gel properties including metrics of polymer length distribution, material stiffness, viscoelasticity or other properties.

Various techniques may be used to lyophilize or freeze-dry the scaffold. Freezing, lyophilization, or freeze-drying may occur while the substrate and gel is in the mold or after removing the substrate and gel from the mold. Lyophilization removes water through sublimation. The lyophilization may occur at any temperature or pressure or for any duration. The temperature, pressure, or duration of lyophilization may be optimized to create a desired porosity of natural material in the scaffold for example a porous collagen. For example, the porosity of collagen may be greater than 80%.

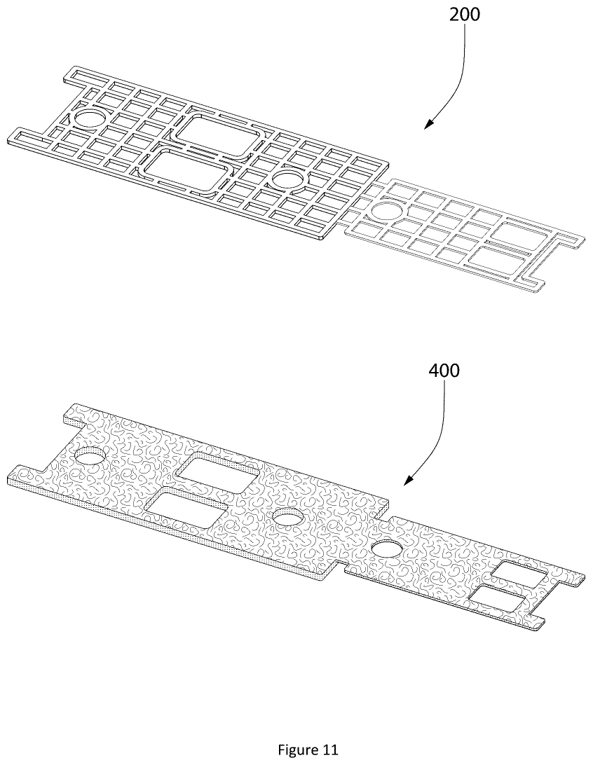

Now referring to FIG. 11, a scaffold 400 may be made of a synthetic material structure or substrate and an integrated natural material. Some of the openings 206 of the synthetic material structure 200 may be filled by the integrated natural polymer material. In some embodiments, the natural polymer material is collagen.

Any additional techniques for processing a natural material may be used during the gelling process or after lyophilization. Additional processes may be used to achieve desired mechanical properties of natural material layers or the scaffold itself. Additional processes may also be used to reduce the degradation rate or achieve any desired degradation rate of natural material layers or the entire scaffold. For example, the natural material may be collagen and techniques to increase collagen crosslinking may be used during gelling or after lyophilization to enhance mechanical properties or reduce the degradation rate of the scaffold.

In some embodiments, a natural material is incorporated into a synthetic material structure by 3D printing. In some embodiments, strands of 3D printed natural material can be oriented in any direction with respect to the synthetic material structure. In some embodiments, a natural material is 3D printed such that it fills in thru-holes, or other gaps in the synthetic material structure. In some embodiments, strands of natural material are printed in a substantially parallel direction such that polymers of the natural material are substantially aligned. In some embodiments, a combination of molding and 3D printing may be used to apply a natural polymer to the synthetic material structure. For example, 3D printing may be used to incorporate natural material strands within a synthetic material structure and molding of a natural polymer may be then used to encapsulate a scaffold. In another example, 3D printing may be used to fill in larger gaps or thru-holes in a synthetic material structure with a natural material, and molding may be used to fill in smaller gaps. In some embodiments, 3D printed natural material layers have different polymer properties than molded natural material layers.

In various embodiments, scaffolds of the present invention can be embedded or conjugated with factors that promote healing, including but not limited to growth factors such as epidermal growth factor (EGF), platelet derived growth factor (PDGF), basic fibroblast growth factor (bFGF), transforming growth factor-β (TGF-β), and tissue inhibitors of metalloproteinases (TIMP). Additional factors can include antibiotics, bacteriocides, fungicides, silver-containing agents, analgesics, and nitric oxide releasing compounds. Scaffolds of the present invention may also be seeded with proteins, enzymes, cells, such as fibroblasts, osteoblasts, keratinocytes, epithelial cells, endothelial cells, mesenchymal stem cells, and/or embryonic stem cells. Scaffolds may be cultured with cells for any amount of time before introduced to a subject or used in a surgical procedure. Natural material polymers may be seeded with cells or other factors before or after being poured into the mold, before or after 3D printing the natural material, or before or after curing or solidification of the natural material gel. The polymers of the scaffold may be actively reorganized by cultured cells before introduced to a subject or used in a surgical procedure. Cells may reorganize the polymers of the scaffold such that alignment, stiffness, density, or other properties of the scaffold are changed. Cells may deposit extracellular matrix proteins or fibers onto or within the scaffold. Cells may secrete ligands, growth factors, or other signaling molecules onto or within the scaffold. In some embodiments, cells are seeded onto a scaffold and cultured for a range of 1 to 4 weeks. In some embodiments, the cells are seeded onto a scaffold and allowed to proliferate until reaching a desired cell density. The scaffold may be soaked in a solution containing a desired factor for incorporation at any step of its fabrication. In some embodiments, cells are incorporated into a natural material polymer before it is cured. In this embodiment, the scaffold is not lyophilized, but rather is incubated with culture medium to support cell viability and/or proliferation. In some examples, osteoblasts may be seeded onto a scaffold in which a natural polymer has pores or micropores with diameter of about 100 μm to promote cell attachment to the scaffold.

Embodiments of the 3D printer may perform electrocompaction on the collagen media, which promotes organization and alignment of the collagen molecules by electrical charge. Advantageously, this increases the bond strength of the collagen membrane during the 3D printing process. In some embodiments, the methods of depositing natural and/or synthetic materials to create the scaffold may comprise aligning the molecules of the materials used. Aligning the molecules may be achieved through electrocompaction, electrospinning, gravity-based alignment, extrusion-based alignment, or any methods known in the art. In some embodiments, aligning the molecules is achieved through electrocompaction. In some embodiments, electrocompaction is performed by the 3D printer. In some embodiments, electrocompaction further comprises generating an electric field between two electrodes across a solution of the scaffold material. As an example, the electric field generates a pH gradient and charges the support material molecules such that they align and compact at an isoelectric point. In some embodiments, the electrodes used may be of any suitable shape depending on the shape of the scaffold. For example, and without limitation, linear, curvilinear, planar, curviplanar, or tubular electrodes may be used. In some embodiments, electrocompaction promotes organization and alignment in the scaffold material, leading to an increase in mechanical strength of the scaffold. Alignment in the scaffold material may create anisotropics in mechanical properties. For example mechanical strength may be stronger in the aligned direction.

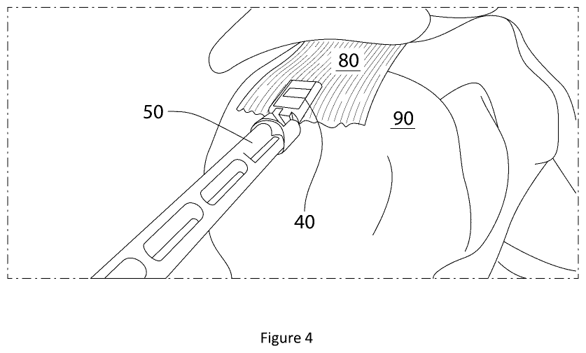

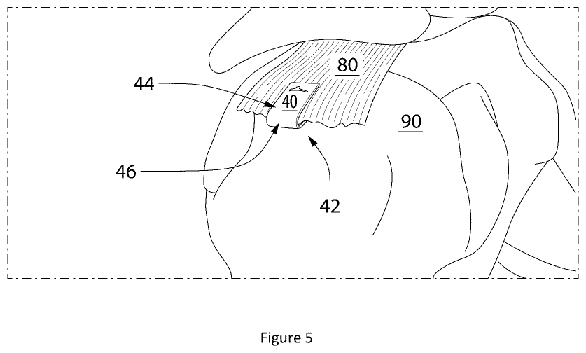

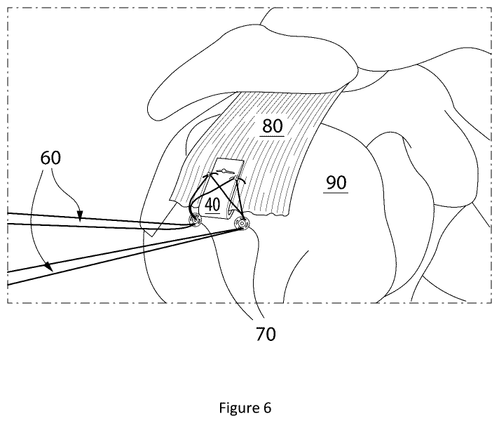

Scaffolds of the present invention can be configured to heal or repair a target site. For example, as shown in FIGS. 4-6, scaffolds of the present invention can be used to wrap around a soft tissue such as a tendon or ligament for secure attachment to a bone surface. In a first step and as shown in FIG. 4, the scaffold and suture anchoring device 50 is loaded with a scaffold 40 in a manner similar to as shown in FIG. 3. Scaffold 40 is positioned about soft tissue 80. As can be seen in FIG. 5, scaffold 40 is uniquely structurally designed such that a thin region 42 of scaffold 40 presents a lower profile when positioned between bone 90 and the bottom surface of soft tissue 80 to minimize lifting of soft tissue 80 from bone 90. Region 46 of scaffold 40 thickens and permits folding around the peripheral edge of soft tissue 80. As shown in FIG. 6, region 44 is thicker and provides enhanced mechanical strength for tying and securing any number of sutures 60 and/or bone anchors 70 needed to properly secure the soft tissue the bone.

Scaffolds of the present invention can be applied to a subject in locations in which tissue growth is desired. For example, scaffolds may be applied at sights of ligament or tendon injury. Scaffolds may be applied at wounds in the skin or other organs to promote healing. Scaffolds may be used as part of a repair construct. For example, scaffolds may promote the attachment of an injured soft tissue to bone. A scaffold may be sutured in one location to a soft tissue and in one location to bone. The scaffold may be designed such that it reduces tearing of sutures through the tissue.

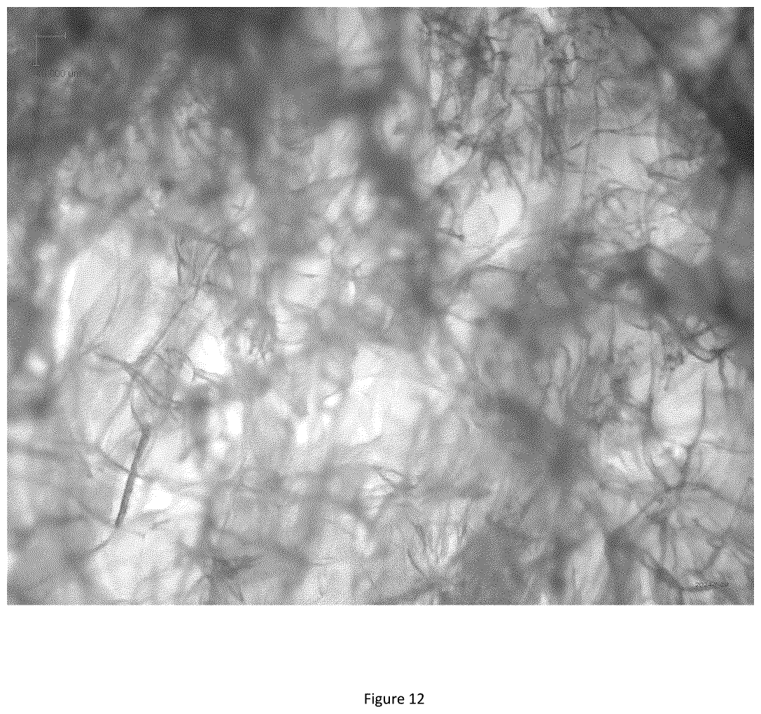

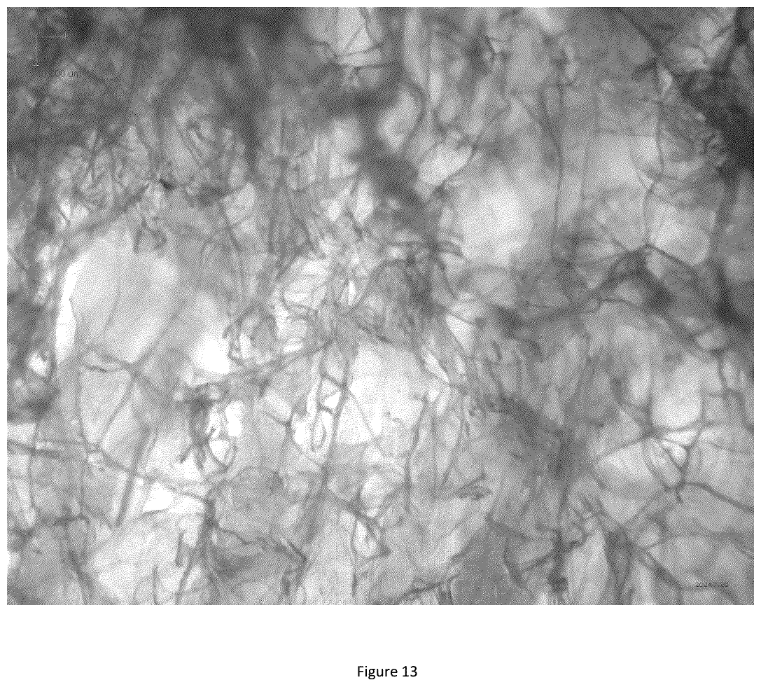

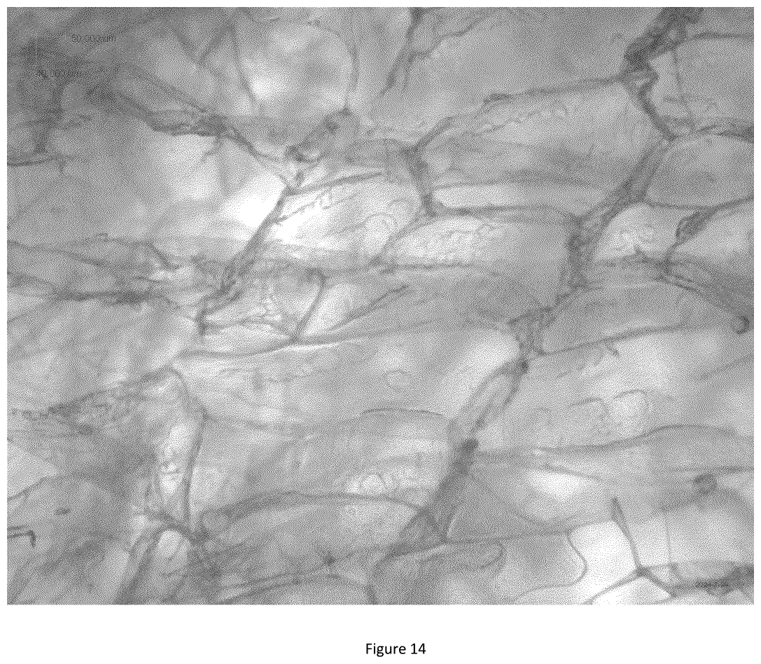

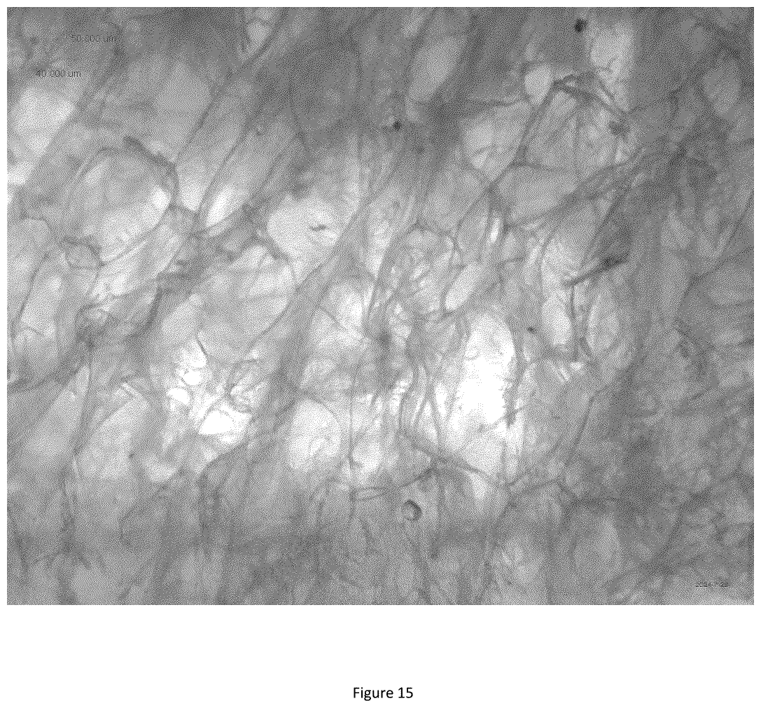

Scaffolds of the present invention may be designed to achieve certain properties including stiffness, clastic modulus, viscoelasticity, toughness, porosity, or degradation rate. Stiffness or elastic modulus may be determined by a tensile test. Scaffolds may be designed to achieve certain properties of the synthetic polymers or natural polymers included in the scaffold including polymer alignment, polymer length, or polymer crosslinking. Properties of the polymers may be measured qualitatively or quantitatively by imaging techniques including brightfield imaging, second harmonic generation imaging, electron microscopy, or fluorescence microscopy. A fluorescent molecule may be incorporated into the scaffold or the polymers of the scaffold to enhance imaging. The scaffold may be imaged in a hydrated state or a lyophilized state. Now referring to FIG. 12 through FIG. 15, in some embodiments, the natural material polymer used in the scaffold is collagen. The collagen may be imaged to qualitatively and quantitatively assess the structure of the collagen and its porosity. In some examples, the images may be annotated to calculate the density of collagen fibers in the image field of view. In some examples, the collagen fibers may be annotated so that metrics of the distribution of collagen fiber lengths in the scaffold can be calculated. In some examples, the images of collagen fibers in images may be annotated to calculate the persistence length of collagen fibers. In some examples, properties of collagen organization measured through imaging are correlated with material properties of the scaffold including stiffness, viscoelasticity, or degradation rate.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A biocompatible scaffold, comprising:
a synthetic polymer substrate having a geometry with a plurality of openings; and
a natural polymer material integrated with the synthetic polymer substrate;
wherein the natural polymer material at least partially fills one or more of the openings in the synthetic polymer substrate.

2. The scaffold claim 1, wherein the synthetic polymer substrate geometry is a mesh, a meshwork, a grid, or a 3D geometry with the plurality of openings forming thru-holes.

3. The scaffold of claim 1, wherein the synthetic polymer substrate is a molded layer, a 3D printed layer, or any combination thereof.

4. The scaffold of claim 1, wherein the natural polymer material encapsulates the synthetic polymer substrate.

5. The scaffold of claim 1, wherein the integrated natural polymer material is a molded layer, a 3D printed layer, an electrocompacted layer, or any combination thereof.

6. The scaffold of claim 1, wherein the synthetic polymer is selected form the group consisting of: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), poly(L-lactic acid) (PLLA), polyglycolic acids (PGA), poly(lactide-co-glycolides) (PLGA), polycaprolactone (PCL), tri-calcium phosphate (TCP), polycaprilactone-tri-calcium phosphate (PCL-TCP), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO), polyorthoesters, and any combinations thereof.

7. The scaffold of claim 1, wherein the natural polymer is selected from the group consisting of: collagen, fibrin, fibrinogen, thrombin, elastin, laminin, fibronectin, hyaluronic acid, chondroitin 4-sulfate, chondroitin 6-sulfate, dermatan sulfate, gelatin, heparin sulfate, heparin, keratan sulfate, proteoglycans, polysaccharides, chitin, chitosan, alginic acids, alginates, and any combinations thereof.

8. The scaffold of claim 1, wherein the porosity of the natural polymer material is at least 80%.

9. A method of making a biocompatible scaffold comprising the steps of:
forming a synthetic polymer substrate having a plurality of openings; and
integrating a natural polymer material into one or more of the openings in the synthetic polymer substrate.

10. The method of claim 9, wherein the forming step comprises molding or 3D printing.

11. The method of claim 9, further comprising lyophilizing the synthetic polymer substrate and integrated natural polymer material.

12. The method of claim 9, wherein the integrating step comprises molding, 3D printing or electrocompacting.

13. The method of claim 9, wherein the integrating step comprises encapsulating the synthetic polymer substrate within the natural polymer material.

14. The method of claim 9, wherein the step of forming the synthetic polymer substrate comprises forming a geometry that is a mesh, a meshwork, a grid, or a 3D geometry with thru-holes.

15. The method of claim 9, wherein the integrating step further comprises:
positioning the synthetic polymer substrate into a mold;
mixing a buffer solution with the natural polymer material to form a gel;
adding the gel into the mold containing the synthetic polymer substrate;
curing the gel;
freezing the synthetic polymer substrate and gel; and
lyophilizing the frozen synthetic polymer substrate and gel to form the scaffold.
